(19) United States
(12) Patent Application Publication
(10) Pub. No.: US 2004/0097481 A1
(43) Pub. Date: May 20, 2004

(54) Water-soluble mesoporphyrin compounds and methods of preparation

(76) Inventors: Benjamin Levinson, Montgomery, NJ (US); George S. Drummond, New York, NY (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/713,889

(22) Filed: Nov. 14, 2003

Related U.S. Application Data
(60) Provisional application No. 60/427,851, filed on Nov. 20, 2002.

(51) Int. Cl. A61K 31/555; A61K 31/4172; A61K 31/405; A61K 31/198

(52) U.S. Cl. 514/185; 514/400; 514/419; 514/561; 514/564; 514/565; 514/567

(57) ABSTRACT

Water soluble tin mesoporphyrin compounds are described. Methods of preparation of water soluble metal mesoporphyrin compounds and pharmaceutical preparations are also described.

### CROSS REFERENCE TO RELATED APPLICATIONS

This application claims the benefit of U.S. Provisional Application No. 60/427,851 filed Nov. 20, 2002, the contents of which are hereby incorporated by reference in their entirety.

### BACKGROUND OF THE INVENTION

The present invention generally relates to water-soluble mesoporphyrin compounds and processes for their preparation. More specifically, one or more embodiments of the invention relate to processes for making novel pharmaceutical compositions containing such compounds and use of said compositions in the treatment of various conditions such as neonatal and other forms of hyperbilirubinemia.

Tin (IV) mesoporphyrin IX chloride or stannsoporfin is a mesoporphyrin chemical compound having the structure indicated in FIG. 1. It has been proposed for use, for example, as medicament in the treatment of various diseases including, for example, psoriasis (U.S. Pat. No. 4,782,049 to Kappas et al.) and infant jaundice (for example, in U.S. Pat. Nos. 4,684,637, 4,657,902 and 4,692,440). Stannsoporfin is also known to inhibit heme metabolism in mammals, to control the rate of tryptophan metabolism in mammals, and to increase the rate at which heme is excreted by mammals (U.S. Pat. Nos. 4,657,902 and 4,692,440 both to Kappas et al.).

Processes for obtaining stannsoporfin are known in the art. Protoporphyrin IX iron (III) chloride or hemin, of the structural formula indicated in FIG. 2, is commonly used as starting material. The hemin is generally hydrogenated to form an intermediate mesoporphyrin IX dihydrochloride, which is subsequently subjected to tin insertion, yielding stannsoporfin.

The above-referenced methods for the preparation of the stannsoporfin, or tin (IV) mesoporphyrin IX, however, result in a non-water soluble compound. Non-water soluble compounds are difficult to use as therapeutic agents, absent special delivery modes, such as encapsulation into a tablet or capsule or via use as a powder. Applications of stannsoporfin in therapeutic treatment of conditions affecting neonates, children, and adults have thus been hindered.

### SUMMARY OF THE INVENTION

One or more embodiments of the present invention provide novel methods for the preparation of water-soluble mesoporphyrin compounds. Specific embodiments provide novel methods for preparing water soluble metal mesoporphyrin compounds. Other embodiments of the present invention provide a novel pharmaceutical composition incorporating a water-soluble tin mesoporphyrin for use in the treatment of various ailments, including neonatal hyperbilirubinemia.

According to one or more embodiments, reaction of tin mesoporphyrin IX dichloride or stannsoporfin with an amino acid in a basic solution forms a novel final compound, a tin mesoporphyrin IX amino acid, such as tin (IV) mesoporphyrin IX arginate. According to one or more embodiments, the final compound can be frozen and vacuum dried so that it can be isolated in a substantially pure, water-soluble, solid form or powder. In one or more embodiments, the substantially pure water-soluble, solid form or powder can be used via injection, orally or by transdermal delivery, such as a transdermal patch, to permit therapeutically useful and active dose volumes.

### BRIEF DESCRIPTION OF THE DRAWINGS

FIG. 1 illustrates the chemical structure of tin mesoporphyrin chloride (tin (IV) mesoporphyrin IX dichloride) or stannsoporfin;

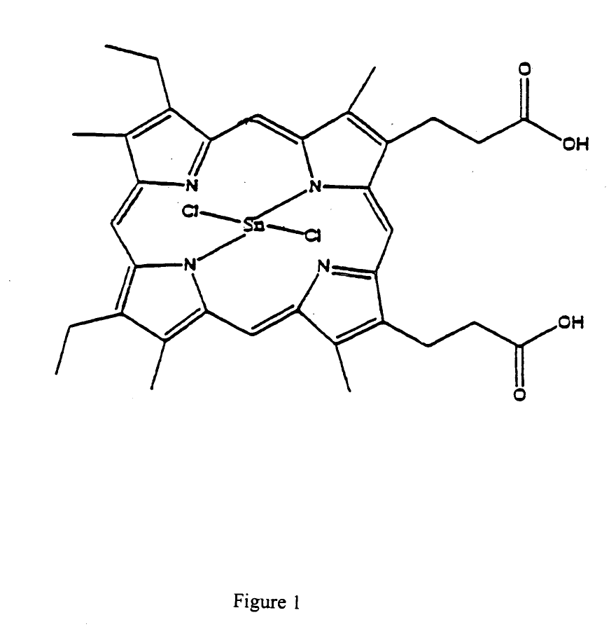

FIG. 2 illustrates the chemical structure of protoporphyrin IX iron (III) chloride or hemin;

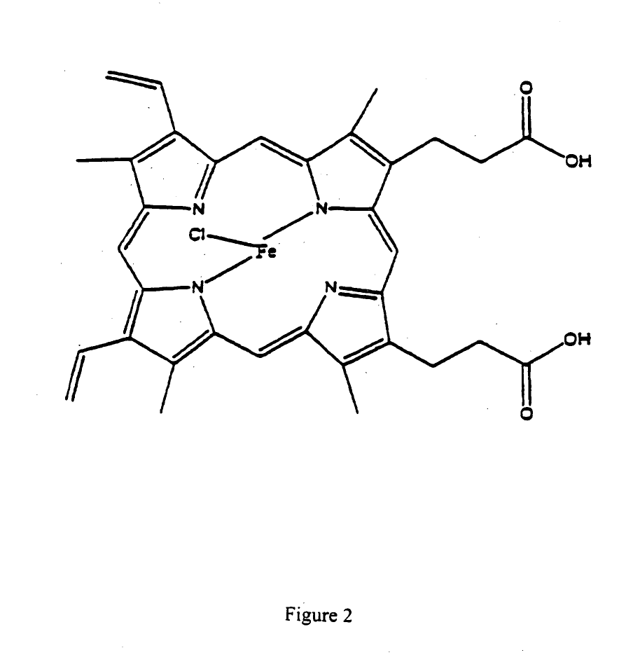

FIG. 3 illustrates the conversion of protoporphyrin IX iron (III) chloride (ferriporphyrin chloride or hemin) to mesoporphyrin IX formate; and

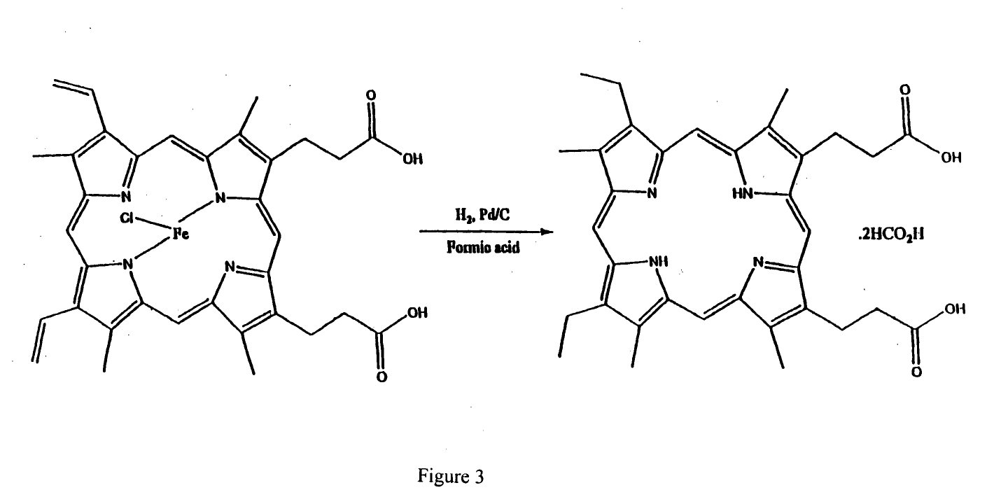

FIG. 4 illustrates the conversion of mesoporphyrin IX formate to tin mesoporphyrin chloride (tin (IV) mesoporphyrin IX dichloride) or stannsoporfin.

### DETAILED DESCRIPTION

It is to be appreciated that the various process parameters described herein (by way of example only, temperature, time, and pressure) are approximations and may be varied, and certain steps may be performed in different order. Before describing several exemplary embodiments of the invention, it is to be understood that the invention is not limited to the details of construction or process steps set forth in the following description. The invention is capable of other embodiments and of being practiced or carried out in various ways.

In overview, according to one or more embodiments, a tin mesoporphyrin compound is reacted with one or more amino acids in a solution such as a basic solution to produce water-soluble amino-acid complexes of tin mesoporphyrin or stannsoporfin. According to one or more embodiments, tin (IV) mesoporphyrin IX (or stannsoporfin) can be obtained according to a variety of methods, for example, through the methods disclosed in copending U.S. patent application Ser. No. 10/453,815, filed on Jun. 3, 2003, which is hereby incorporated herein by reference. However, it will be understood that other methods can be used to produce mesoporphyrin halides such as tin mesoporphyrin IX dichloride, and the present invention is not limited to a particular method of mesoporphyrin production.

According to one or more embodiments, a two-stage hydrogenation process is used to prepare tin mesoporhyrin. In the first stage, a reaction mixture of hemin and a hydrogenation catalyst are subjected to a first elevated temperature for a first period of time. In certain embodiments, the first stage temperature can be in the range of about 85-95° C. and the period of time is at least about one hour, for example, between about 1-3 hours.

In a second stage of hydrogenation, the reaction mixture is cooled to a second temperature for a second period of time. For example, the second temperature can be in a range of about 45-50° C. and hydrogenated for a second period of time of about 3-6 hours, in order to convert substantially all hemin (protoporphyrin IX iron (III) chloride) to mesoporphyrin IX formate. In certain embodiments, this second stage can also be conducted in the presence of formic acid. The same catalyst may be used as in the first step described above, so that the two stages of the process may be conducted in the same reactor. Optionally, a further charge of hydrogen may be supplied to the reactor prior to commencing the second stage. According to one or more embodiments, the second hydrogenation stage increases the yield of the mesoporphyrin IX formate, while reducing the amount of impurities in the final metal mesoporphyrin halide.

By the method described above, the mesoporphyrin IX intermediate compound in the present invention is not isolated as a dihydrochloride, but rather as a formate salt. It will be understood, of course, that other processes can be used for the preparation of tin (IV) mesoporphyrin intermediates.

The mesoporphyrin IX formate may be isolated from a formic acid solution by the addition of a solvent such as ether or other organic solvent, leading directly to the mesoporphyrin IX formate intermediate, which is further subjected to drying. Ethers such as, for example, methyl tert-butyl ether, diethyl ether or di-isopropyl ether, among others, may be used. One specific embodiment of the invention involves the use of methyl tert-butyl ether.

According to the process described above, less solvent is required compared to other processes, and such smaller volumes allow for less filter time to obtain the intermediate. In exemplary embodiments, ratios of the amount of hemin to the amount of solvent of about 1:10 to about 1:20 may be used. In addition, the filtration and washings of the mesoporphyrin IX formate are rapid. After drying, a crude intermediate formate is obtained, in high yields (about 80-95%) and its purity, established by HPLC, is about or above 97%.

The insertion of metal into mesoporphyrin IX formate to obtain metal mesoporphyrin halide is described below with specific reference to tin, to prepare stannsoporfin.

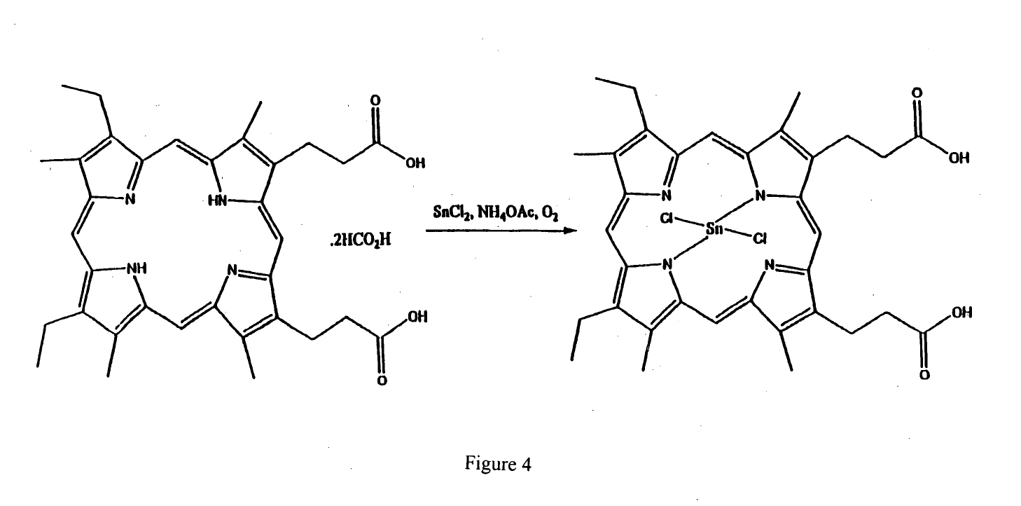

According to an embodiment of the invention, the insertion of tin into mesoporphyrin IX formate is illustrated in FIG. 4. In one or more embodiments, mesoporphyrin IX formate is subjected to heating with a tin (II) carrier in an acid such as acetic acid, buffered with an acetate ion, in the presence of an oxidant, at reflux. Tin (II) carriers such as tin (II) halides or tin (II) acetate can be used. Suitable acetate counter ions include ammonium, sodium or potassium ions. Oxidants such as oxygen from air or in pure form as well as hydrogen peroxide can also be used. In one exemplary embodiment of the invention, the insertion of metal into mesoporphyrin IX formate occurs. In one or more embodiments, mesoporphyrin IX formate is subjected to heating with tin (II) chloride in acetic acid, buffered with ammonium acetate, and the reaction is conducted in the presence of air, at reflux. According to this embodiment, tin mesoporphyrin dichloride is isolated from the reaction mixture by the addition of water, followed by filtration to provide a filter cake. Still according to the exemplary embodiment, prior to drying at about 90-100° C., the filter cake is triturated into hot, dilute hydrochloric acid, for example, at a concentration of about 0.1 N-6N, at about 90-100° C. According to this embodiment, the crude, substantially pure tin mesoporphyrin chloride (crude tin (IV) mesoporphyrin IX dichloride) is obtained with a yield of about 75-95% and a purity of about 95%, as judged by HPLC analysis.

In accordance with at least one embodiment, the tin mesoporphyrin IX dichloride obtained by the above-described process may be further purified by dissolving the product in an aqueous inorganic base solution, for example, dilute ammonium hydroxide, followed by treatment with charcoal. The product is then re-precipitated by addition to an acid solution, such as acetic acid, hydrochloric acid or a mixture thereof. The above dissolving, charcoal treatment and re-precipitation steps may be repeated a number of times, typically about 1-3 times in order to ensure the desired purity. Prior to drying, the cake is triturated in hot, dilute hydrochloric acid of a concentration of about 0.1N-6N, at a temperature of about 90-100° C., in order to remove any residual ammonium salts. The tin mesoporphyrin chloride product (tin (IV) mesoporphyrin IX dichloride or stannsoporfin) is obtained in a yield of about 50-70%, with an HPLC purity of about or greater than 97%.

The process described above may also be performed to produce substantially pure or pharmaceutical quality tin mesoporphyrin chloride (tin (IV) mesoporphyrin IX dichloride or stannsoporfin) in large scale quantities, such as quantities exceeding about 0.1 kg through and including multiple kilogram amounts, by slight modifications of the above procedure, such as increased reaction or drying times as appropriate based upon the increase in scale of the starting reactants. Temperature and pressure times likewise can be modified as needed within the scope of this invention. The tin mesoporphyrin chloride product (tin (IV) mesoporphyrin IX dichloride or stannsoporfin) is obtained in the large scale production process in a yield of about 60-90%, with an HPLC purity of about 97%.

According to one or more embodiments of the present invention, tin mesoporphyrin, such as the tin (IV) mesoporphyrin IX obtained as described above, is then reacted with one or more amino acids in a solution such as a basic solution to produce water-soluble amino-acid complexes of tin (IV) mesoporphyrin IX or stannsoporfin. The amino acid selected may be one or more of the known amino acids, including but not limited to arginine, glycine, alanine, leucine, serine, and lysine. The basic solutions may comprise any common base in aqueous form, including, but not limited to, sodium hydroxide, trisodium phosphate, an hydroxide of an alkali metal (Group IIA), an hydroxide of an alkaline earth metal (Group IIA) or amines such as ethanol amine or an aqueous solution of one or more of said bases.

The water soluble compounds of the present invention can be prepared and administered in a wide variety of oral and parenteral dosage forms. Thus, the compounds of the present invention can be administered by injection, that is, intravenously, intramuscularly, intrathecally, intracutaneously, subcutaneously, intraduodenally, or intraperitoneally. Also, the compounds of the present invention can be administered by inhalation, for example, intranasally. Additionally, the compounds of the present invention can be administered transdermally. Moreover, the compounds of the present invention can be administered rectally, vaginally, or across any mucosal surface, such as for example the buccal mucosal of the mouth. It will be obvious to those skilled in the art that the following dosage forms may comprise as the active component, either a compound of FIG. I or a corresponding pharmaceutically acceptable salt of a compound of FIGS. I or II.

According to one or more embodiments of the invention, the preparation of pharmaceutical compositions can involve the use of pharmaceutically acceptable carriers, which can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, an encapsulating material, or drug delivery agents, such as liposomal preparations.

In embodiments including powders, the carrier is a finely divided solid which is in a mixture with the finely divided active component. In embodiments including tablets, the active component is mixed with the carrier having the necessary binding properties in suitable proportions and compacted in the shape and size desired.

The powders and tablets preferably contain from about 0.1 to about 50 percent of the active compound. Suitable carriers include, but are not limited to, magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, such as natural or synthetic gums, resins, methyl cellulose, sodium carboxymethyl cellulose, and other well-known suspending agents.

One or more embodiments of the invention include solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from about 0.1 to about 50 mg, preferably 0.1 to about 10 mg according to the particular application and the potency of the active component and size of the patient. The composition can, if desired, also contain other compatible therapeutic agents.

According to one or more embodiments, in therapeutic use as agents for treating neonatal hyperbilirubinemia, the compounds utilized in the pharmaceutical methods of this invention are administered at the initial dosage of about 0.1 mg to about 20 mg per kilogram body weight (IM) daily. Specific exemplary embodiments involve the use of about 0.5 mg to about 5 mg per kilogram body weight (IM) for the treatment of neonatal hyperbilirubinemia. The dosages, however, may be varied depending upon the requirements of the patient, the severity of the condition being treated and the compound being employed. Determination of the proper dosage for a particular situation is within the skill of the art. In one embodiment, generally, treatment is initiated with smaller dosages which are less than the optimum dose of the compound. Thereafter, the dosage is increased by small increments until the optimum effect under the circumstance is reached.

Exemplary embodiments of the invention will be further described for illustrative purposes with reference to the following non-limiting examples.

### EXAMPLE 1

### Preparation of Tin (IV) Mesoporphyrin IX Arginate Salt

A)—Preparation of mesoporphyrin IX formate—A 2000 ml hydrogenation vessel was charged with 40.0 g hemin, 4.0 g 5% Pd/C (50% water by weight), and 800 ml 96% formic acid. Since hemin and mesoporphyrin IX formate as well as all reaction intermediates are reportedly light sensitive materials, care was taken throughout this entire procedure to minimize the exposure of the reaction to visible or ultraviolet light.

The vessel was flushed with a nitrogen flow for 10 minutes. With vigorous stirring, it was then pressurized to 50 psi with hydrogen for ten minutes, depressurized, and the cycle repeated. The vessel was further pressurized to 50 psi with hydrogen and the temperature was raised to 90° C. over approximately 20 minutes.

The hydrogenation reaction was maintained at 90° C. and 45-55 psi for 1-1.5 hours. The reaction mixture was not stable for extended periods of time at 90° C. The time at this temperature was sufficient to dissolve all hemin and convert the majority of this material to the intermediate and final product, mesoporphyrin IX formate. The reaction was cooled to 50° C./50 psi over 20 minutes. The pressure and temperature were maintained for 3 hours. The reaction mixture was shown to be stable at this temperature for up to 18 hours. The reaction was cooled to 20-25° C., de-pressurized, and flushed with nitrogen.

The catalyst was removed by filtration through a bed of 20 g celite to produce a filter cake. The filter cake was rinsed with 3×50 ml formic acid, and the filtrate including formic acid and the filter cake was charged to a 2000 ml three-necked, round-bottom flask equipped with a magnetic stir bar, thermometer, and distillation bridge. The formic acid solvent was distilled off under aspirator vacuum to a residual volume of 200 ml. The distillation bridge was replaced with an addition funnel. With moderate agitation, 800 ml methyl tert-butyl ether was added drop wise over 30-60 minutes. The resultant suspension was agitated at 20-25° C. for 60 minutes prior to cooling to −20 to −25° C. for 1 to 2 hours. The suspension was filtered under reduced pressure. The filter cake was rinsed with 100 ml filtrate, followed by 2×50 ml methyl tert-butyl ether and dried under high vacuum at 40-60° C. for 24 hours. About 30-38 g of mesoporphyrin IX formate were obtained (yield of 75-95%).

B)—Preparation of Substantially Pure Tin Mesoporphyrin Chloride (tin (IV) mesoporphyrin IX dichloride or stannsoporfin). A dark 1000 ml three-necked, round-bottom flask equipped with a mechanical stirrer, condenser, bubbler, and an aeration tube was charged with 30.0 g mesoporphyrin IX formate, 34.5 g tin (II) chloride, 7.1 g ammonium acetate, and 600 ml acetic acid. The suspension was stirred at 20-25° C. for 30 minutes. Mesoporphyrin IX formate and tin mesoporphyrin as well as all reaction intermediates are reportedly light sensitive materials therefore care was taken throughout this entire procedure to minimize the exposure of the reaction to light.

The reaction was warmed to reflux, with aeration, for 3 to 4 hours. The reaction was shown to be stable at 110-115° C. for up to 48 hours. Once complete, the reaction mixture was cooled to 60-70° C. and 300 ml water was added while cooling to 20-25° C. over 60 minutes. The suspension was filtered under reduced pressure. The filter cake was rinsed with 2×60 ml water. A dark, 1000 ml, three-neck, round-bottom, flask equipped with a stir bar, thermometer, condenser, and nitrogen purge was charged with the wet cake from the above step, and 500 ml 1 N HCl. The resultant suspension was warmed to 90° C. for 1 hour. The suspension was filtered under reduced pressure. The filter cake was rinsed with 2×50 ml 0.1N HCl and dried under high vacuum at 80-90° C. for 24 hours. About 25 to 28 g of crude, substantially pure (about or exceeding 95% purity) tin mesoporphyrin chloride (tin (IV) mesoporphyrin IX dichloride or stannsoporfin) was obtained for a yield of about 83-93%.

C)—Preparation of the Arginate Salt. The tin (IV) mesoporphyrin IX dichloride prepared according to the above referenced process is then combined with a solution of arginine in aqueous sodium hydroxide and mixed for a period of sufficient time such that the reaction is closer to completion. The ratio of the tin (IV) mesoporphyrn IX dichloride to the arginine is about 2:1. The ratio of the tin mesoporphyrin IX dichloride to the aqueous sodium hydroxide is about 1:3. The solution is then filtered, rinsed with deionized water and frozen. Following freezing of the filtrate solution including the liquid and the tin mesoporphyrn-amino acid complex, the frozen solution is vacuum dried to provide in a lyophilized product.

### EXAMPLE 2

### Preparation of an Injectible or Transdermal Formulation of Tin (IV) Mesoporphyrin IX Arginate Salt

A)—Preparation of mesoporphyrin IX formate—A 2000 ml hydrogenation vessel was charged with 40.0 g hemin, 4.0 g 5% Pd/C (50% water by weight), and 800 ml 96% formic acid. Since hemin and mesoporphyrin IX formate as well as all reaction intermediates are reportedly light sensitive materials, care was taken throughout this entire procedure to minimize the exposure of the reaction to visible or ultraviolet light.

The vessel was flushed with a nitrogen flow for 10 minutes. With vigorous stirring, it was then pressurized to 50 psi with hydrogen for ten minutes, depressurized, and the cycle was repeated. The vessel was further pressurized to 50 psi with hydrogen and the temperature was raised to 90° C. over approximately 20 minutes.

The hydrogenation reaction was maintained at 90° C. and 45-55 psi for 1-1.5 hours. The reaction mixture was not stable for extended periods of time at 90° C. The time at this temperature was sufficient to dissolve all hemin and convert the majority of this material to the intermediate and final product, mesoporphyrin IX formate. The reaction was cooled to 50° C./50 psi over 20 minutes. The pressure and temperature were maintained for 3 hours. The reaction mixture was shown to be stable at this temperature for up to 18 hours. The reaction was cooled to 20-25° C., de-pressurized, and flushed with nitrogen.

The catalyst was removed by filtration through a bed of 20 g celite. The filter cake was rinsed with 3×50 ml formic acid and the filtrate was charged to a 2000 ml three-necked, round-bottom flask equipped with a magnetic stir bar, thermometer, and distillation bridge. The formic acid solvent was distilled off under aspirator vacuum to a residual volume of 200 ml. The distillation bridge was replaced with an addition funnel. With moderate agitation, 800 ml methyl tert-butyl ether was added drop wise over 30-60 minutes. The resultant suspension was agitated at 20-25° C. for 60 minutes prior to cooling to −20 to −25° C. for 1 to 2 hours. The suspension was filtered under reduced pressure. The filter cake was rinsed with 100 ml filtrate, followed by 2×50 ml methyl tert-butyl ether and dried under high vacuum at 40-60° C. for 24 hours. About 30-38 g of mesoporphyrin IX formate was obtained (yield of 75-95%).

B)—Preparation of Substantially Pure Tin Mesoporphyrin Chloride (tin (IV) mesoporphyrin IX dichloride or stannsoporfin). A dark 1000 ml three necked, round-bottom flask equipped with a mechanical stirrer, condenser, bubbler, and an aeration tube was charged with 30.0 g mesoporphyrin IX formate, 34.5 g tin (II) chloride, 7.1 g ammonium acetate, and 600 ml acetic acid. The suspension was stirred at 20-25° C. for 30 minutes. Mesoporphyrin IX formate and tin mesoporphyrin as well as all reaction intermediates are reportedly light sensitive materials therefore care was taken throughout this entire procedure to minimize the exposure of the reaction to light.

The reaction was warmed to reflux, with aeration, for 3 to 4 hours. The reaction was shown to be stable at 110-115° C. for up to 48 hours. Once complete, the reaction mixture was cooled to 60-70° C. and 300 ml water were added while cooling to 20-25° C. over 60 minutes. The suspension was filtered under reduced pressure. The filter cake was rinsed with 2×60 ml water. A dark, 1000 ml, three-neck, round-bottom, flask equipped with a stir bar, thermometer, condenser, and nitrogen purge was charged with the wet cake from the above step, and 500 ml 1N HCI. The resultant suspension was warmed to 90° C. for 1 hour. The suspension was filtered under reduced pressure. The filter cake was rinsed with 2×50 ml 0.1N HCI and dried under high vacuum at 80-90° C. for 24 hours. About 25 to 28 g of crude, substantially pure (about or exceeding 95% purity) tin mesoporphyrin chloride (tin (IV) mesoporphyrin IX dichloride or stannsoporfin) was obtained for a yield of about 83-93%.

C)—Preparation of the Arginate Salt. The tin (IV) mesoporphyrin IX dichloride prepared according to the process described above, is combined with an excess solution of arginine in aqueous sodium hydroxide (the ratio being about 2:1:3) and mixed for a sufficient period of time as to affect dissolution. The ratio of the tin (IV) mesoporphyrin IX dichloride to the arginine is about 2:1. The ratio of the tin mesoporphyrin IX dichloride to the aqueous sodium hydroxide is 1:3. The solution is then filtered, rinsed with deionized water and frozen. Following freezing of the solution, the frozen solution is vacuum dried to result in a lyophilized product.

We expect that the reconstituted product can be resolubilized into DI H₂O or 5% saline, or into one of other known in the art injectible or transdermal solutions, and delivered to the patient by such injectible or transdermal methods. Those skilled in the art would readily appreciate that other amino acids would similarly react with tin (IV) mesoporphyrin IX dichloride to form a water soluble complex consistent with this invention.

For example, we have prepared and reacted a number of amino acids with tin-mesoporphyrin in the presence of NaOH. The material isolated from these reactions have been examined by ¹H NMR and UV/VIS (ultraviolet and visible) spectroscopy, as well as comparing the solubilities of the reaction products have also been compared to the solubilities of their respective starting materials, to determine whether an amino acid-tin-mesoporphyrin complex forms form such a reaction.

Comparison of the ¹H NMR spectra of the starting materials to the reaction products essentially reveals a 1:1 mixture of amino acid and tin-mesoporphyrin. Since the ¹H NMR methodology as explained in the art indicates that ¹H NMR spectroscopy may not be sensitive enough to detect the formation of the desired complexes, we also utilized UV/VIS spectroscopy as an analytical method. The UV/VIS spectroscopy revealed small discrete changes in the spectrum that are likely consistent with the formation of a complex between amino acid and tin-mesoporphyrin.

Solubility profile changes likewise suggest the formation of a novel species, the amino acid—tin-mesoporphyrin complex, upon mixing the tin mesoporhyrin and an amino acid in the presence of NaOH due to a change in the solubility of the amino acids component of the reaction mixture, as indicated in the table below (Methanol=MeOH; Isopropyl alcohol=IPA; Ethyl Acetate=EtOAc; tetra hydrofuran=THF; Methy-t-butylether=MTBE:

| Compound | Water | MeOH | IPA | EtOAc | THF | MTBE |
|---|---|---|---|---|---|---|
| Tin mesoporphyrin | Y | Y | S | N | N | N |
| sodium | | | | | | |
| L-alanine sodium | Y | Y | N | N | N | N |
| L-leucine sodium | Y | N | N | N | N | N |
| L-serine sodium | Y | N | N | N | N | N |
| L-arginine sodium | Y | N | N | N | N | N |
| L-glycine sodium | Y | N | N | N | N | N |
| L-alanine-complex | Y | Y | N | N | N | N |
| L-leucine complex | Y | Y | N | N | N | N |
| L-serine-complex | Y | Y | N | N | N | N |
| L-arginine-complex | Y | Y | N | N | N | N |
| L-lysine complex | Y | Y | N | N | N | N |
| L-glycine-complex | Y | Y | N | N | N | N |

We further reacted a number of additional amino acids with tin mesoporhyrin in the presence of NaOH in accordance with the present invention. The material isolated from these reactions has been examined by ¹H NMR and UV/VIS spectroscopy. The solubilities of these additional reaction products have also been compared to the solubilities of their respective starting materials.

The amino acids L-histidine, L-phenylalanine and L-tyrosine are different from the amino acids reacted so far in that they each contain an aromatic moiety. A π-π interaction between the respective aromatic moieties of the porphyrin and said amino acids produces an obvious chemical shift change in the ¹H NMR spectra of the complexes. The ¹H NMR spectra of the complexes exhibit a significant up-field shift in the tin mesoporphyrin ¹H NMR resonances found at −10.5 ppm. In the complexes, these resonances are found at 9.5 ppm. Examination of the UVIVIS spectra also shows significant changes in the absorption found at ˜350 nm. These absorption changes indicate the likely formation of chemical bonds between the amino acid and the tin mesoporphyrin, further evidencing a formation of a complex between the amino acid and tin mesoporphyrin.

| | | | | | | |
|---|---|---|---|---|---|---|
| Compound | Water | MeOH | IPA | EtOAc | THF | MTBE |

Starting Materials

| | | | | | | |
|---|---|---|---|---|---|---|
| Tin | Y | Y | S | N | N | N |
| mesoporphyrin | | | | | | |
| sodium | | | | | | |
| L-alanine | Y | Y | N | N | N | N |
| sodium | | | | | | |
| L-leucine | Y | N | N | N | N | N |
| sodium | | | | | | |
| L-lysine sodium | Y | N | N | N | N | N |
| L-glycine | Y | N | N | N | N | N |
| sodium | | | | | | |
| L-histidine | Y | N | N | N | N | N |
| L-phenylalanine | Y | N | N | N | N | N |
| L-tyrosine | S | S | S | N | N | N |

Complexes

| | | | | | | |
|---|---|---|---|---|---|---|
| L-alanine-complex | Y | Y | N | N | N | N |
| L-leucine-complex | Y | Y | N | N | N | N |
| L-serine-complex | Y | Y | N | N | N | N |
| L-arginine-complex | Y | Y | N | N | N | N |
| L-lysine-complex | Y | Y | N | N | N | N |
| L-glycine-complex | Y | Y | N | N | N | N |
| L-histidine-complex | Y | Y | N | N | N | N |
| L-phenylalanine- | Y | Y | N | N | N | N |
| complex | | | | | | |
| L-tyrosine-complex | Y | Y | N | N | N | N |

Finally, we reacted all naturally known amino acids with tin mesoporphyrin in accord with the present invention. The amino acids were allowed to react with tin mesoporphyrin in the presence of NaOH in water. The material isolated from these reactions were examined by ¹H NMR and UV/VIS spectroscopy. The solubilities of the reaction products were then also compared to the solubilities of their respective starting materials.

| | | | | | | |
|---|---|---|---|---|---|---|
| Compound | Water | MeOH | IPA | EtOAc | THF | MTBE |

Starting Materials

| | | | | | | |
|---|---|---|---|---|---|---|
| Tin | Y | Y | S | N | N | N |
| mesoporphyrin | | | | | | |
| sodium | | | | | | |
| L-alanine | Y | Y | N | N | N | N |
| sodium | | | | | | |
| L-leucine | Y | N | N | N | N | N |
| sodium | | | | | | |
| L-serine sodium | Y | N | N | N | N | N |
| L-arginine | Y | N | N | N | N | N |
| sodium | | | | | | |
| L-lysine sodium | Y | N | N | N | N | N |
| L-glycine | Y | N | N | N | N | N |
| sodium | | | | | | |
| L-histidine | Y | N | N | N | N | N |
| sodium | | | | | | |
| L-phenylalanine | Y | N | N | N | N | N |
| sodium | | | | | | |
| L-tyrosine | Y | S | N | N | N | N |
| sodium | | | | | | |
| DL-tryptophan | Y | Y | N | N | N | N |
| sodium | | | | | | |
| L-proline | | | | | | |
| sodium | | | | | | |

Complexes

| | | | | | | |
|---|---|---|---|---|---|---|
| L-alanine-complex | Y | Y | N | N | N | N |
| L-leucine-complex | Y | Y | N | N | N | N |
| L-serine-complex | Y | Y | N | N | N | N |
| L-arginine-complex | Y | Y | N | N | N | N |
| L-lysine-complex | Y | Y | N | N | N | N |
| L-glycine-complex | Y | Y | N | N | N | N |
| L-histidine-complex | Y | Y | N | N | N | N |
| L-phenylalanine- | Y | Y | N | N | N | N |
| complex | | | | | | |
| L-tyrosine-complex | Y | Y | N | N | N | N |
| DL-tryptophan- | Y | Y | N | N | N | N |
| complex | | | | | | |
| L-proline-complex | | | | | | |

A number of the reaction products exhibit different solubility properties when compared to the solubility properties of their respective starting materials. As a result, a change in the solubility of the amino acids component of the reaction mixture suggests and supports formation of a tin-mesoporphyrin-amino acid complex. Accordingly, using the change in solubility properties of the amino acids as a guide, the above table suggests and supports complex formation between tin mesoporphyrin and all the amino acids listed. We expect similar complex formation with tin mesoporphyrin and the amino acid proline based on the other amino acid results as tabulated.

For many of the reaction products, differences ranging from subtle to obvious were observed between the UV/VIS spectra of tin mesoporphyrin sodium and the reaction products. This change is suggestive of the formation of a new chemical species. For the reaction products derived from the amino acids glycine sodium, alanine sodium, leucine sodium, lysine sodium and serine sodium, a shoulder appears on the peak at ˜400 nm in the UV/VIS spectrum. A more dramatic change is observed for the reaction products derived from the amino acids histidine sodium, phenylalanine sodium, tyrosine sodium, tryptophan sodium, methionine sodium and threonine sodium, where an altogether new absorbance is observed at ˜380 nm in the UV/VIS spectrum. The UV/VIS spectrum for the reaction product derived from arginine sodium remains essentially unchanged when compared to the UVNIS spectrum of tin mesoporphyrin sodium. Thus, it is likely that the biochemical and therapeutic properties of tin-mesoporphyrin would be exhibited in the novel tin-mesoporphyrin—amino acid complexes formed by the present invention.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A water soluble tin mesoporphyrin compound comprising tin mesoporphyrin complexed with a second agent.

2. The water soluble tin mesoporphyrin compound of claim 1, wherein the second agent includes an amino acid.

3. The water soluble tin mesoporphyrin compound of claim 2, wherein the compound is in liquid or solid form.

4. The water soluble tin mesoporphyrin compound of claim 2, wherein the amino acid is selected from the group consisting of arginine, glycine, alanine, leucine, serine, lysine, histidine, phenylalanine, tyrosine and combinations thereof.

5. A pharmaceutical formulation comprising a water soluble tin mesoporphyrin compound and at least one pharmaceutically acceptable carrier.

6. The pharmaceutical formulation of claim 5, wherein the water soluble tin mesoporphyrin compound comprises tin mesoporphyrin complexed with a second agent.

7. The pharmaceutical formulation of claim 6, wherein the second agent includes an amino acid.

8. The pharmaceutical formulation of claim 7, wherein the water soluble compound is in liquid or solid form.

9. The pharmaceutical formulation of claim 7, wherein the amino acid is selected from the group consisting of arginine, glycine, alanine, leucine, serine, lysine, histidine, phenylalanine, tyrosine and combinations thereof.

10. The pharmaceutical formulation of claim 7, wherein the formulation contains between about 0.1 and about 50 mg of tin mesoporphryin.

11. A method of preparing a water soluble complex of tin mesoporphyrin comprising mixing a tin mesoporphyrin compound in solution with an amino acid.

12. The method of claim 11, wherein the solution is a basic solution.

13. The method of claim 12, wherein the solution comprises an aqueous solution of sodium hydroxide.

14. The method of claim 2, wherein the amino acid is selected from the group consisting of arginine, glycine, alanine, leucine, serine, lysine, histidine, phenylalanine, tyrosine and combinations thereof.

15. The method of claim 11, wherein the ratio of the tin mesoporphyrin compound to amino acid is at least about 2:1.

16. The method of claim 14, wherein the ratio of the tin mesoporphyrin to basic solution is at least about 1:3.

17. The method of claim 11, further comprising filtering the solution to obtain a solid or a pharma acceptable liquid.

18. The method of claim 17, wherein when the filtered product is a solid, further comprising vacuum drying the solid.

19. The method of claim 11, wherein the tin mesoporphyrin compound includes a mesoporphyrin halide.

20. The method of claim 19, wherein the halide includes tin mesoporphyrin dichloride.

21. The method of claim 11, further comprising subjecting a tin mesoporphyrin intermediate to a catalytic hydrogenation, recovering a formate salt of tin mesoporphyrin, drying the formate salt to obtain a tin mesoporphyrin formate, subjecting the mesoporphyrin Ix formate to a chemical metal insertion process reaction with a metal halide compound under buffered, reaction conditions to produce a metal mesoporphyrin halide.

22. A pharmaceutical formulation including a tin mesoporphyrin compound formed by the method of claim 11 mixed with at least one pharmaceutically acceptable carrier.

23. A method of preparing a water-soluble complex of metal mesoporphyrin, which comprises:
subjecting a reaction mixture of hemin and a hydrogenation catalyst for a first elevated temperature and a first period of time;
subjecting the reaction mixture to a second elevated temperature for a second period of time;
recovering a formate salt from the reaction mixture and drying the salt to obtain a metal mesoporphyrin IX formate;
subjecting the mesoporphyrin IX formate to a chemical metal insertion process reaction with a metal halide compound under reaction conditions to produce a metal mesoporphyrin halide; and reacting the metal mesoporphyrin halide with at least one amino acid in the presence of a basic solution to produce a water-soluble complex of metal mesoporphyrin.

24. The method of claim 23, wherein the first temperature is higher than the second temperature.

25. The method of claim 24, wherein the first temperature is between about 85-95° C.

26. The method of claim 25, wherein the reaction mixture of hemin and hydrogenation catalyst is in an acid and subjected to hydrogen pressure for at least one hour.

27. The method of claim 26, wherein the second temperature is between about 45-50° C. and the second period of time is at least about 3 hours.

28. The method of claim 27, wherein subjecting the mesoporphyrin IX formate to a chemical metal insertion process reaction with a metal halide compound is in the presence of an oxidant under buffered, acidic reaction conditions.

29. A pharmaceutical composition including a metal mesoporphyrin compound made by the process of claim 23.

30. The pharmaceutical composition of claim 29, wherein the metal mesoporphyrin includes tin mesoporphyrin.

31. The method of claim 30, wherein the amino acid is selected from the group consisting of arginine, glycine, alanine, leucine, serine, lysine, histidine, phenylalanine, tyrosine and combinations thereof.

32. A method of treating a human being with a medical condition comprising administering a pharmaceutically effective amount of a water soluble tin mesoporphyrin compound.

33. The method of claim 32, wherein the water soluble tin mesoporhyrin compound comprises tin mesoporphyrin complexed with a second agent.

34. The method of claim 33, wherein the second agent includes an amino acid.

35. The method of claim 34, wherein the amino acid is selected from the group consisting of arginine, glycine, alanine, leucine, serine, lysine, histidine, phenylalanine, tyrosine and combinations thereof.

36. The method of claim 35, wherein the condition is hyperbilirubinemia.

37. The method of claim 35, wherein the condition is psoriasis.
